(19) United States
(12) Patent Application Publication
(10) Pub. No.: US 2021/0154436 A1
(43) Pub. Date: May 27, 2021

(54) INTRODUCER NEEDLE AND RELATED DEVICES, SYSTEMS, AND METHODS

(71) Applicant: Becton, Dickinson and Company, Frank Lakes, NJ (US)

(72) Inventors: Yiping Ma, Layton, UT (US); David Stockey, Holly Springs, NC (US); Christopher Anthony Basciano, Apex, NC (US); Marcus Rademacher, Fuquay Varina, NC (US)

(21) Appl. No.: 17/093,481

(22) Filed: Nov. 9, 2020

Related U.S. Application Data
(60) Provisional application No. 62/939,311, filed on Nov. 22, 2019.

(51) Int. Cl. A61M 25/06; A61M 25/02

(52) U.S. Cl. CPC A61M 25/0606; A61M 25/02; A61M 2025/0293; A61M 2025/0213; A61M 25/0612

(57) ABSTRACT

A catheter system to reduce a risk of transfixing a blood vessel. The catheter system may include a catheter adapter, which may include a proximal end, a distal end, and a lumen extending therebetween. The catheter system may include a catheter extending distally from the distal end of the catheter adapter. The catheter system may include an introducer needle extending through the catheter. The introducer needle may include a sharp distal tip and a primary bevel extending from an outer edge of the introducer needle to the sharp distal tip. The outer edge may be parallel to a central axis of the introducer needle and aligned with the sharp distal tip. The primary bevel may include a primary bevel angle with respect to the central axis. The primary bevel angle may be between 20° and 33°.

### RELATED APPLICATIONS

The application claims the benefit of U.S. Provisional Patent Application No. 62/939,311, filed Nov. 22, 2019, and entitled NEEDLE TIP DESIGNS WITH SHORTENED BEVEL LENGTH, which is incorporated herein in its entirety.

### BACKGROUND

A catheter is commonly used to infuse fluids into vasculature of a patient. For example, the catheter may be used for infusing normal saline solution, various medicaments, or total parenteral nutrition.

The catheter may include a peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter system. Once placement of the introducer needle has been confirmed, the clinician may remove the introducer needle, leaving the catheter in place for future fluid infusion.

During catheter insertion, the blood vessel may be transfixed when the clinician advances the introducer needle too far. In further detail, the blood vessel may be transfixed by the introducer needle if the introducer needle enters the top of the blood vessel and accidentally punctures the bottom of the blood vessel too. Transfixing the blood vessel may be painful to the patient and may increase a risk of hemorrhage, hematoma, infection, and tissue damage. Further, fluid and medicaments may be infused into an extravascular space instead of into the blood vessel. In response to the blood vessel being transfixed, the introducer needle may be withdrawn, and the clinician may perform an additional needle stick, leading to decreased patient satisfaction.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a vascular access device, as well as related systems and methods. More particularly, the present disclosure relates to an introducer needle and related devices, systems, and methods. In some embodiments, a catheter system to reduce a risk of transfixing a blood vessel may include a catheter adapter. In some embodiments, the catheter system may include a catheter adapter, which may include a proximal end, a distal end, and a lumen extending therebetween.

In some embodiments, the catheter system may include a catheter extending distally from the distal end of the catheter adapter. In some embodiments, the catheter system may include an introducer needle extending through the catheter. In some embodiments, the introducer needle may include a sharp distal tip and a primary bevel extending from an outer edge of the introducer needle to the sharp distal tip. In some embodiments, the outer edge may be parallel to a central axis of the introducer needle and aligned with the sharp distal tip. The primary bevel may include a primary bevel angle with respect to the central axis. The primary bevel angle may be between 20° and 33°.

In some embodiments, the primary bevel angle may be 22.2°. In some embodiments, the introducer needle may include a first cut opposing a second cut. In some embodiments, the first cut may correspond to a secondary bevel and the second cut may correspond to a tertiary bevel. In some embodiments, the first cut may be planar, and the second cut may be planar. In some embodiments, the first cut and the second cut may extend downwardly from the primary bevel. In some embodiments, the first cut may intersect a plane of the primary bevel at a first intersection line and/or the second cut may intersect the plane of the primary bevel at a second intersection line. In some embodiments, the first cut may meet the second cut at another line, which may include the sharp distal tip. In some embodiments, the first cut and/or the second cut may be disposed at a secondary angle.

In some embodiments, the secondary angle may be between 18° and 26°. In some embodiments, the secondary angle may be 22°. In some embodiments, the primary bevel angle may be 22.2°. In some embodiments, the catheter may include a peripheral intravenous catheter.

In some embodiments, a method for reducing a risk of blood vessel transfixion may include inserting the introducer needle of the catheter system into vasculature of a patient. In some embodiments, in response to the introducer needle being inserted into the vasculature, the method may include withdrawing the introducer needle from the catheter system.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1 is an upper perspective view of an example needle assembly, illustrating an example introducer needle, according to some embodiments;

FIG. 2 is a top cross-sectional view of an example catheter system, according to some embodiments;

FIG. 3 is an enlarged upper perspective view of a distal end of the catheter system, according to some embodiments;

FIG. 4 is a side view of an example distal end of the introducer needle of the catheter system, in accordance with some embodiments;

FIG. 5A is a cross-sectional view of the distal end of the introducer needle, taken along the line 5-5 of FIG. 4, in accordance with some embodiments;

FIG. 5B is a 30/60 isometric view of the distal end of the introducer needle, according to some embodiments;

FIG. 5C is a distal end view of the introducer needle, illustrating the isometric view of FIG. 5B, according to some embodiments;

FIG. 6 is an enlarged top view of an example bevel of the introducer needle, in accordance with some embodiments;

FIG. 7A is a side view of an example distal end of the introducer needle, according to some embodiments;

FIG. 7B is a side view of another example distal end of the introducer needle, according to some embodiments;

FIG. 8 is a side view of another example distal end of the introducer needle, according to some embodiments; and

FIG. 9 is a table illustrating a relationship between needle tip geometry, global peak force, and initial peak force, in accordance with some embodiments.

### DESCRIPTION OF EMBODIMENTS

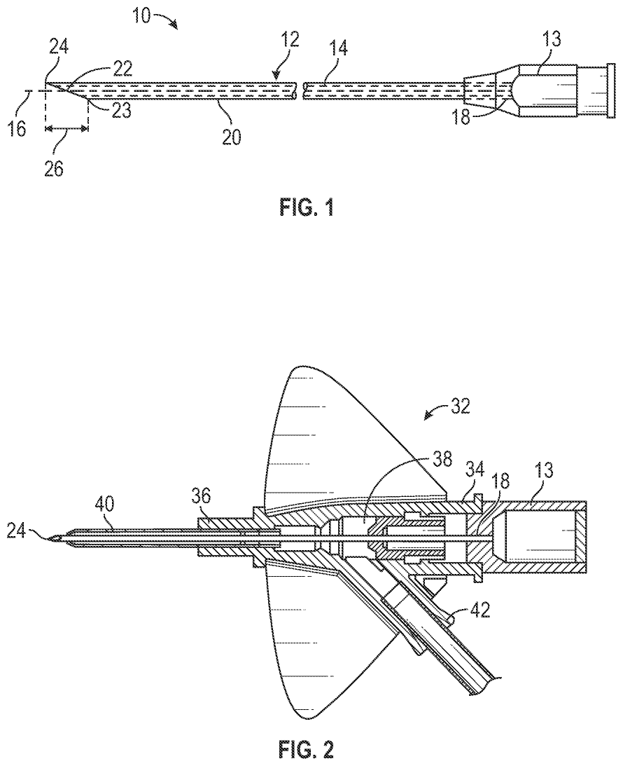

Referring now to FIG. 1, in some embodiments, a vascular access device 10 may include a needle 12. In some embodiments, the needle 12 may be configured to reduce a risk of blood vessel transfixion. In some embodiments, the needle 12 may also facilitate blood vessel penetration such that a force to penetrate skin and a blood vessel of a patient with the needle 12 is not substantially increased beyond that of traditional needle tips.

As illustrated in FIG. 1, in some embodiments, the needle 12 may extend distally from a needle hub 13. In some embodiments, the needle 12 may include a shaft 14, which may be cylindrical or generally cylindrical. In some embodiments, a central axis 16 may extend through a center of the shaft 14. In some embodiments, a proximal end 18 of the needle 12 or the shaft 14 may be secured within the needle hub 13. In some embodiments, the proximal end 18 of the needle 12 may include an opening, which may facilitate blood flow into the needle hub 13 for flashback and/or blood collection purposes. In some embodiments, the shaft 14 may include an outer edge 20, which may be disposed along a length of the shaft 14 to the proximal end 18.

In some embodiments, the distal end of the needle 12 may include a primary bevel 22 and a sharp distal tip 24 disposed at a distal end of the primary bevel 22. In some embodiments, the sharp distal tip 24 may facilitate entry of the needle 12 and an over-the-needle catheter into vasculature of the patient. In some embodiments, the primary bevel 22 may include a length 26, which may extend from a distal end 23 of the outer edge 20 to the sharp distal tip 24 and is parallel to the central axis 16. In some embodiments, the outer edge 20 may be aligned with the sharp distal tip 24 and the central axis 16. In some embodiments, the outer edge 20 may be disposed on an opposite side of the needle 12 as the sharp distal tip 24.

As discussed in more detail below, in some embodiments, the length 26 of the primary bevel 22 may be short, which may decrease a risk of blood vessel transfixion and decrease a force utilized by the clinician to insert the needle 12 into the blood vessel. In some embodiments, the primary bevel 22 may be angled to increase fluid flow through the sharp distal tip 24.

Referring now to FIG. 2, in some embodiments, the needle 12 may be included in a catheter system for reducing a risk of blood vessel transfixion. In some embodiments, the catheter system may include a catheter adapter 32, which may include a proximal end 34, a distal end 36, and a lumen 38 extending therebetween. In some embodiments, the catheter adapter 32 may include a side port 42 in fluid communication with the lumen 38. In this manner, some embodiments of the side port 42 may be configured to receive an extension set to facilitate infusion and/or blood withdrawal through a catheter 40.

In some embodiments, the catheter 40 may extend distally from the distal end 36 of the catheter adapter 32. In some embodiments, the needle hub 13 may be coupled to the proximal end 34 of the catheter adapter 32 such that the needle 12 extends through catheter 40. In some embodiments, the primary bevel 22 and the sharp distal tip 24 may thus be disposed distal to a distal end of the catheter 40 to pierce the skin and vasculature of the patient. In some embodiments, the needle 12 may include an introducer needle, configured to introduce the catheter 40 into the vasculature. In some embodiments, a gauge and/or type of the needle 12 may vary.

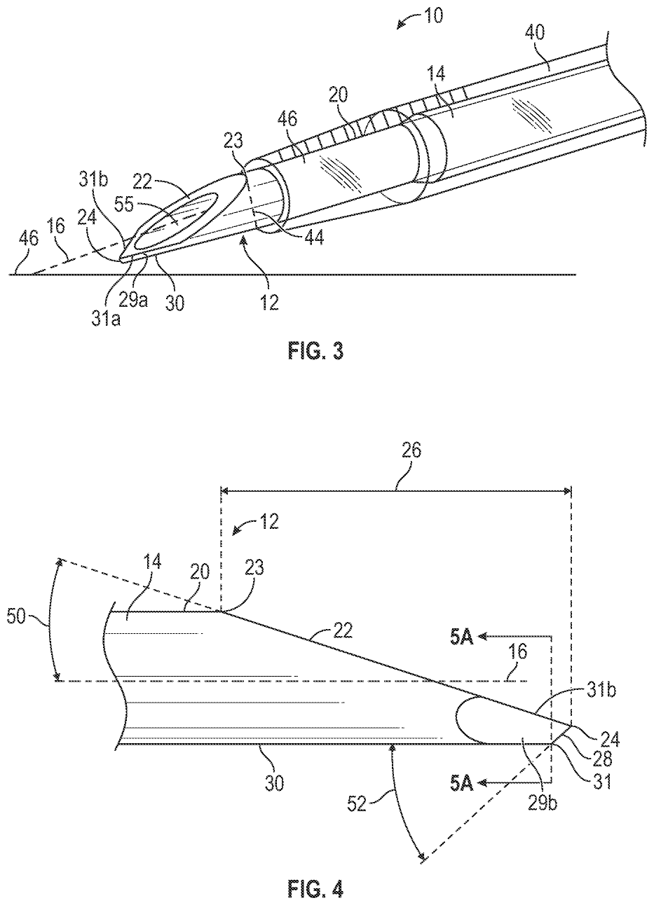

Referring now to FIG. 3, in some embodiments, the shaft 14 of the needle 12 may be longitudinally disposed along the central axis 16 between the proximal end 18 (see, for example, FIGS. 1-2) and the distal end 23 of the outer edge 20. In this manner, in some embodiments, the shaft 14 may have a length corresponding to a distance between the proximal end 18 and the distal end 23 of outer edge 20. In some embodiments, a distal end 44 of the shaft 14 may be proximate the primary bevel 22. In some embodiments, the primary bevel 22 of the needle 12 may extend from the distal end 23 of the outer edge 20 of the shaft 14 to the sharp distal tip 24. In some embodiments, the length of the shaft 14 may extend through the catheter 40 such that the primary bevel 22 of the needle 12 and the sharp distal tip 24 may be exposed at the distal end of the catheter 40.

In some embodiments, the primary bevel 22 may be configured to point away from the skin of the patient and towards a top of a catheter system, as illustrated in FIG. 3. Thus, the opposing outer edge 30 may be closest portion of the catheter system to the skin 46 of the patient during insertion of the needle 12 into the patient.

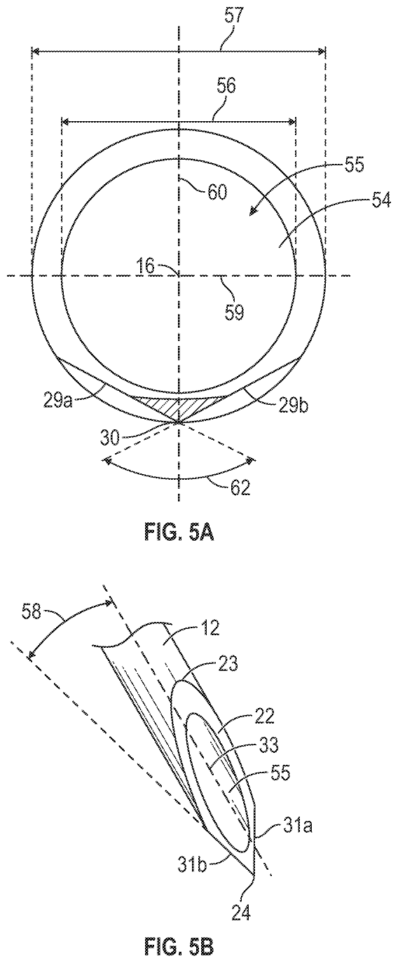

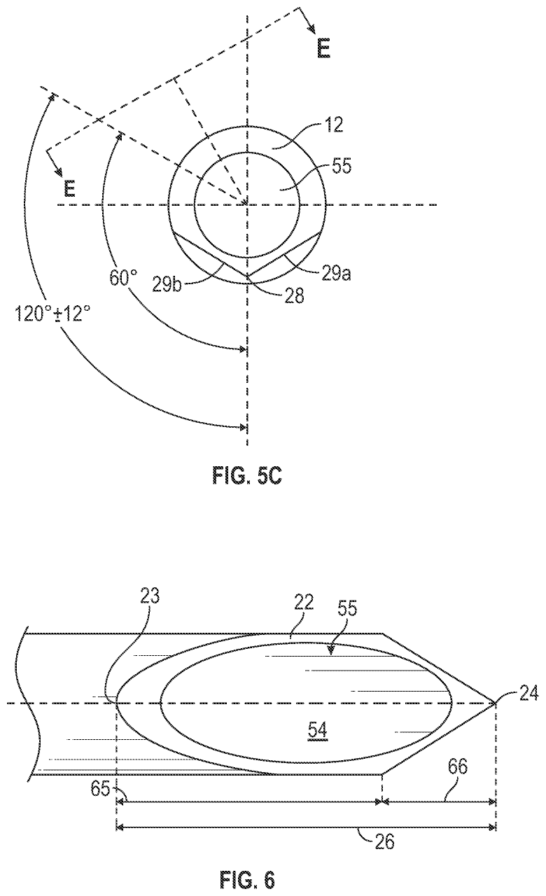

Referring now to FIG. 4, in some embodiments, the primary bevel 22 may include the length 26 extending between the distal end 23 of the outer edge 20 and the sharp distal tip 24 and parallel to the central axis 16. In some embodiments, the distal end of the needle 12 may include a line 28 where a first cut 29a and a second cut 29b may meet (see also, FIGS. 3 and 5). In some embodiments, the line 28 may include a straight edge, which may facilitate cutting. In some embodiments, the first cut 29a may be similar or identical to the second cut 29b on an opposing side of the needle 12.

In some embodiments, the primary bevel 22 may be straight or generally straight. In some embodiments, the needle 12 may include an opposing outer edge 30, which may oppose or be directly opposite the outer edge 20. In some embodiments, the opposing outer edge 30 may extend distal to the shaft 14 and to the line 28. In some embodiments, a length of the line 28 may extend from the sharp distal tip 24 proximally to a distal end 37 of the opposing outer edge 30. In some embodiments, the opposing outer edge 30 may be aligned with the central axis 16 and the sharp distal tip 24.

In some embodiments, the primary bevel 22 may include a primary bevel angle 50, which may be measured with respect to the central axis 16. In some embodiments, the length 26 of the primary bevel 22 may be determined by the primary bevel angle 50 such that the length 26 may be reduced in response to the primary bevel angle 50 being increased. In some embodiments, the primary bevel angle 50 and the length 26 that results from the primary bevel angle 50 may reduce a likelihood of blood vessel transfixion upon insertion of the needle 12 into a patient's vasculature. In some embodiments, given a same diameter of a patient's vein, a particular primary bevel 22 with a shorter length 26 would reduce a likelihood of the sharp distal tip 24 touching a back wall of the vein when the clinician observes the flashback indicating venous punctuation and during catheter advancement following venipuncture.

In some embodiments, the line 28 may include an angle 52, which may be measured with respect to the opposing outer edge 30. In some embodiments, the angle 52 may facilitate a sharpness of the sharp distal tip 24. In some embodiments, reducing the angle 52 may increase the sharpness of the sharp distal tip 24. Similarly, in some embodiments, reducing the angle 52 may reduce the needle penetration force required to insert the needle 12 into a patient's vasculature, thereby reducing insertion pain.

FIG. 5A illustrates a cross-section of the needle 12 taken along the line 5-5 depicted in FIG. 4. In some embodiments, the needle 12 may include a lumen 54 extending through the needle 12. In some embodiments, the needle 12 may include a distal opening 55 surrounded by the primary bevel 22. In some embodiments, the distal opening 55 may be formed in the primary bevel 22 to provide fluid communication between the lumen 54 and an external environment.

In some embodiments, the shaft 14 may include an inner diameter 56 and an outer diameter 57. In some embodiments, an outer surface of the needle 12 may include the first cut 29a and the second cut 29b (which may be referred to in the present disclosure as “cuts 29”), which may facilitate insertion of the needle 12 into the patient and increase the sharpness of the needle 12. In some embodiments, the first cut 29a and the second cut 29b may form cutaway portions from the outer diameter 57, which may be generally circular. In some embodiments, the first cut 29a and/or the second cut 29b may be planar and separated by an angle 62, as illustrated, for example, in FIG. 5A. In other embodiments, the first cut 29a and/or the second cut 29b may be curved.

In some embodiments, the first cut 29a may oppose the second cut 29b. In some embodiments, the first cut 29a may correspond to a secondary bevel and the second cut 29b may correspond to a tertiary bevel. In some embodiments, the first cut 29a and the second cut 29b may extend downwardly from the primary bevel 22. In some embodiments, the first cut 29a may intersect a plane of the primary bevel 22 at a first intersection line 31a and/or the second cut 29b may intersect the plane of the primary bevel 22 at a second intersection line 31b. In some embodiments, the first cut 29a may meet the second cut 29b at the line 28, which may include the sharp distal tip 24. In some embodiments, the first cut 29a and the second cut 29b may each extending proximally from the primary bevel 22 to the opposing outer edge 30 directly opposing the outer edge 20.

In some embodiments, a secondary angle 58 may be measured as illustrated in FIGS. 5B-5C. In some embodiments, the secondary angle 58 may correspond to an angle between the first intersection line 31a and the reference line 33 in the 30/60 isometric view. In some embodiments, the secondary angle 58 may also correspond to an angle between the second intersection line 31b and the reference line 33 in the 30/60 isometric view. In some embodiments, reducing the secondary angle 58 may increase the sharpness of the sharp distal tip 24. Similarly, in some embodiments, reducing the secondary angle 58 may reduce the needle penetration force required to insert the needle 12 into a patient's vasculature, thereby reducing insertion pain.

In some embodiments, the inner diameter 56 of the shaft 14 and/or the outer diameter 57 of the shaft 14 may be constant along all or a portion of a length of the shaft 14. In some embodiments, the cuts 29 may reduce a thickness of a wall of the needle 12 at the primary bevel 22 such that the thickness is less than a thickness of a wall of the shaft 14. In some embodiments, the cuts 29 may be disposed below a midline 59 or on a side of the needle 12 configured to be closest to the skin of the patient during insertion into the vasculature. In some embodiments, the thickness of the wall of the shaft 14 may be reduced below the midline 59, which may intersect an intersecting axis 60 dividing the cross-section into four equal quadrants. In some embodiments, the central axis 16 may run perpendicular to the midline 59 and the intersecting axis 60.

Referring now to FIG. 6, in some embodiments, the length 26 of the primary bevel 22 may be measured from the distal end 23 of the outer edge 20 to the sharp distal tip 24. In some embodiments, the distal opening 55 may include an oval shape, a polygonal shape, a circular shape, or any other suitable shape.

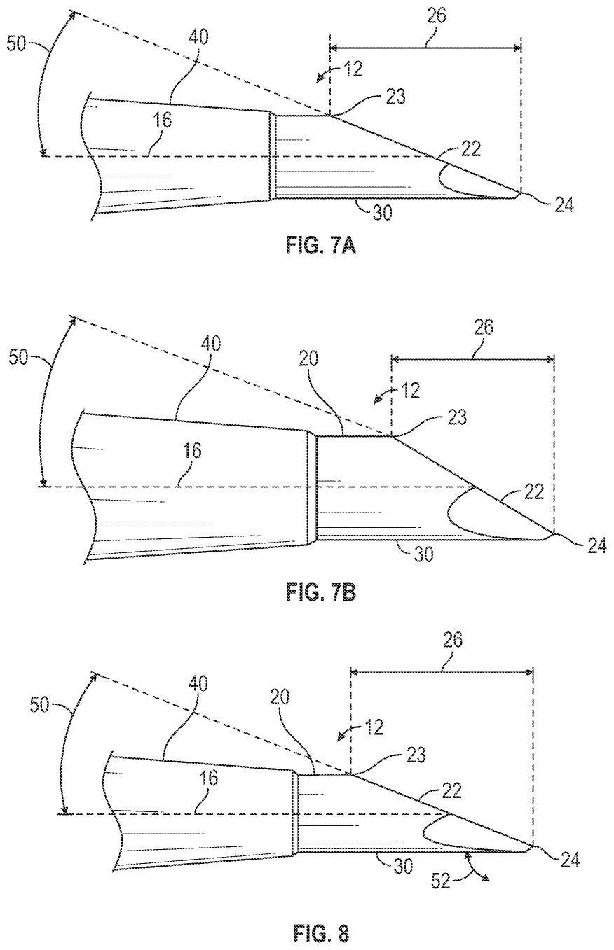

Referring now to FIG. 7A, in some embodiments, the primary bevel angle 50 may be between 20° and 33°, which may reduce the length 26 and a risk of transfixing the blood vessel during insertion of the needle 12 into the blood vessel. In some embodiments, the primary bevel angle 50 may be 22.2°, as illustrated in FIG. 7A, for example. As illustrated in FIG. 7B, in some embodiments, the primary bevel angle 50 may be 31.4°.

Referring now to FIG. 8, in some embodiments, the primary bevel angle 50 may be between 20° and 33°, and the secondary angle 58 may be between 18° and 26°, which may reduce the needle penetration force required to insert the needle 12 may be reduced, thereby also reducing associated insertion pain. In some embodiments, the primary bevel angle 50 may be 22.2°, and the secondary angle 58 may be 22°, as illustrated in FIG. 8, for example.

In some embodiments, a method for reducing a risk of blood vessel transfixion may include inserting the needle 12 of the catheter system into vasculature of the patient. In some embodiments, in response to the needle 12 being inserted into the vasculature, the method may include withdrawing the needle 12 from the catheter system.

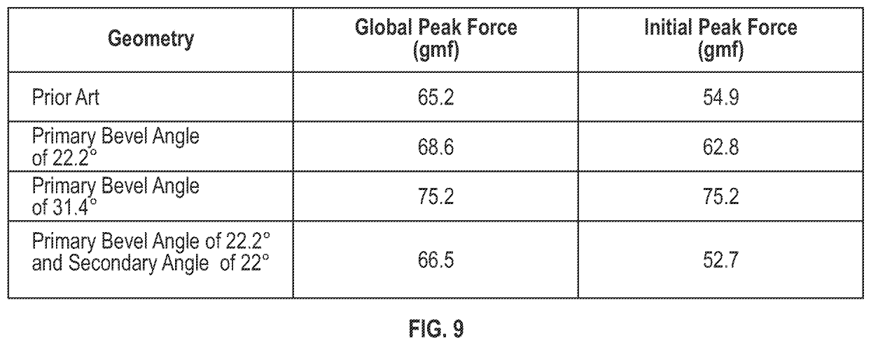

Referring now to FIG. 9, in some embodiments, a primary bevel angle 50 between 20° and 33° greatly reduced a risk of transfixing the blood vessel and also unexpectedly only increased a global peak force and an initial peak force a small amount compared to the prior art. In some embodiments, a combination of a primary bevel angle 50 between 20° and 33° and a secondary angle 58 between 18° and 26° greatly reduced the risk of transfixing the blood vessel and also unexpectedly decreased the initial peak force compared to the prior art. The global peak force and the initial peak force may be measured during insertion of the needle 12 into the blood vessel of the patient. Lower global peak forces and initial peak forces are desired to facilitate insertion of the needle 12 into the blood vessel by the clinician.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A catheter system to reduce a risk of transfixing a blood vessel, the catheter system comprising:
a catheter adapter comprising a proximal end, a distal end, and a lumen extending therebetween;
a catheter extending distally from the distal end of the catheter adapter; and
an introducer needle extending through the catheter, the introducer needle comprising:
a sharp distal tip; and
a primary bevel extending from an outer edge of the introducer needle to the sharp distal tip, wherein the outer edge is parallel to a central axis of the introducer needle and aligned with the sharp distal tip, wherein the primary bevel comprises a primary bevel angle with respect to the central axis, wherein the primary bevel angle is between 20° and 33°.

2. The catheter system of claim 1, wherein the primary bevel angle is 22.2°.

3. The catheter system of claim 1, wherein the introducer needle further comprises:
a first cut and a second cut each extending proximally from the primary bevel to an opposing outer edge directly opposing the outer edge, wherein the opposing outer edge is parallel to the central axis and the outer edge.

4. The catheter system of claim 3, wherein the secondary angle is between 18° and 26°.

5. The catheter system of claim 3, wherein the secondary angle is 22°.

6. The catheter system of claim 5, wherein the primary bevel angle is 22.2°.

7. The catheter system of claim 1, wherein the first cut is planar and the second cut is planar.

8. An introducer needle, comprising:
a sharp distal tip; and
a primary bevel extending from an outer edge of the introducer needle to the sharp distal tip, wherein the outer edge is parallel to a central axis of the introducer needle and aligned with the sharp distal tip, wherein the primary bevel comprises a primary bevel angle with respect to the central axis, wherein the primary bevel angle is between 20° and 33°.

9. The introducer needle of claim 8, wherein the primary bevel angle is 22.2°.

10. The introducer needle of claim 8, further comprising a first cut and a second cut extending proximally from the primary bevel to an opposing outer edge directly opposing the outer edge, wherein the opposing outer edge is parallel to the central axis and the outer edge.

11. The introducer needle of claim 10, wherein the secondary angle is between 18° and 26°.

12. The introducer needle of claim 10, wherein the secondary angle is 22°.

13. The introducer needle of claim 12, wherein the primary bevel angle is 22.2°.

14. The introducer needle of claim 8, wherein the first cut is planar and the second cut is planar.

15. A method for reducing a risk of blood vessel transfixion, comprising:
inserting an introducer needle of a catheter system into vasculature of a patient, wherein the catheter system comprises:
a catheter adapter comprising a proximal end, a distal end, and a lumen extending therebetween;
a catheter extending distally from the distal end of the catheter adapter; and
an introducer needle extending through the catheter, the introducer needle comprising:
a sharp distal tip; and
a primary bevel extending from an outer edge of the introducer needle to the sharp distal tip, wherein the outer edge is parallel to a central axis of the introducer needle and aligned with the sharp distal tip, wherein the primary bevel comprises a primary bevel angle with respect to the central axis, wherein the primary bevel angle is between 20° and 33°; and
in response to the introducer needle being inserted into the vasculature, withdrawing the introducer needle from the catheter system.

16. The method of claim 15, wherein the primary bevel angle is 22.2°.

17. The method of claim 15, wherein the introducer needle further comprises:
a first cut and a second cut extending proximally from the primary bevel to an opposing outer edge directly opposing the outer edge, wherein the opposing outer edge is parallel to the central axis and the outer edge.

18. The method of claim 17, wherein the secondary angle is between 18° and 26°.

19. The method of claim 17, wherein the secondary angle is 22°.

20. The method of claim 19, wherein the primary bevel angle is 22.2°.
